(19) United States
(12) Patent Application Publication
(10) Pub. No.: US 2010/0124743 A1
(43) Pub. Date: May 20, 2010

(54) METHOD FOR DIAGNOSIS OF CANCER

(75) Inventor: Tomonori Nagaoka, Tokyo (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: OLYMPUS CORPORATION, Tokyo (JP)

(21) Appl. No.: 12/524,140

(22) PCT Filed: Jan. 23, 2008
(86) PCT No.: PCT/JP2008/050909

§ 371 Date: Jul. 22, 2009

(30) Foreign Application Priority Data
Jan. 23, 2007 (JP) 2007-012471

(51) Int. Cl. C12Q 1/68

(52) U.S. Cl. 435/6

(57) ABSTRACT

Disclosed is a method for diagnosing cancer with high accuracy through quantification of cancer cell-derived DNA, which comprises the steps of: (1) extracting free DNA from a plasma collected from a test subject; (2) quantifying the extracted free DNA and calculating the free DNA content per unit volume of the plasma to obtain a first calculation value; (3) comparing the first calculation value with a second threshold value which is equal to or higher than a first threshold value; and (4) determining that the test subject is highly unlikely affected by cancer if the first calculation value is lower than the first threshold value, determining that the test subject is likely affected by cancer if the first calculation value is equal to or higher than the first threshold value and lower than the second threshold value, or determining that the plasma used for the quantification is contaminated by normal cell-derived DNA if the first calculation value is equal to or higher than the second threshold value.

### TECHNICAL FIELD

The present invention relates to a method for detecting cancer which uses free DNA in plasma.

Priority is claimed on Japanese Patent Application No. 2007-012471, filed Jan. 23, 2007, the content of which is incorporated herein by reference.

### BACKGROUND ART

Conventionally, methods for detecting a tumor marker which shows up in blood have been used as one of the cancer diagnosis methods. The tumor marker means a cancer cell-derived protein freed from cancerous cells through disruption. In conventional cancer diagnosis methods, it is determined that the test subject is likely affected by cancer if the quantitative value of the tumor marker in blood is equal to or higher than a certain value.

On the other hand, it is known that not only proteins but also DNA are freed into blood through disruption of cancerous cells in the same manner. Reportedly, in comparison between healthy subjects and cancer patients, the quantity of cancer cell-derived free DNA in blood is significantly higher in cancer patients than in healthy subjects. Therefore, it is considered that the quantification of the cancer cell-derived free DNA in a body fluid such as blood can enable the diagnosis of whether or not the test subject is affected by cancer. As such cancer diagnosis methods, there are proposed, for example: a method comprising the steps of detecting that the test subject is likely affected by cancer if a nucleic acid having a length of 200 by or greater is detected in a body fluid or a body excretion by amplification through polymerase chain reaction (PCR) or the like, and further performing a mutation assay on the nucleic acid (see Patent Document 1); and a method comprising the steps of quantifying a genomic DNA derived from cellular debris contained in a body fluid or the like, and performing an assay on the DNA if the quantitative value is above a predetermined value (see Patent Document 2).

Incidentally, even if the diagnosis has proven that the test subject is affected by cancer, only the quantification of a nucleic acid in a body fluid is not able to specify the organ where the cancer occurs. In addition, it is known that, if cancer occurs, mutation(s) occur in a specific nucleic acid due to the organ of cancer occurrence. Accordingly, identification of the type of nucleic acid mutation may possibly be able to specify the organ of cancer occurrence. Here, the nucleic acid mutation can be exemplified by DNA point mutation and structural abnormalities such as chromosome deletion and amplification. For example, it is known that point mutations of the K-ras gene occur in about 70% of pancreatic cancers. In addition, Loss of Heterozygosity (hereunder, abbreviated as LOH) analyses have reported that deletions occur in specific chromosome arms to various types of cancers. For example, frequent LOH in the short arm of chromosome 3 in lung cancer is known. Moreover, multiplication of the long arm of chromosome 8 and RB2 amplification in breast cancer are known.

Patent Document 1: U.S. Pat. No. 6,143,529

Patent Document 2: United States Patent Published Application No. 2004/0259101A1

### DISCLOSURE OF INVENTION

However, for example, even if an attempt is made to quantify cancer cell-derived free DNA in whole blood, a large amount of normal lymphocyte-derived DNA may also be contained in the whole blood while the amount of the cancer cell-derived DNA is very small. Accordingly, it is difficult to quantify the cancer cell-derived free DNA by direct extraction of DNA from the whole blood. Therefore, for example, a method can be considered in which not the whole blood but the plasma separated from the whole blood is used to extract and quantify the cancer cell-derived free DNA in the plasma. However, even in this method, contamination by normal cell-derived, such as lymphocyte-derived, DNA may occur depending on the DNA extraction method, and therefore not the cancer cell-derived free DNA but the normal cell-derived DNA might be quantified, which leads to misdiagnosis of cancer. In addition, in such a case where contamination occurs by the normal cell-derived DNA, the detection sensitivity for gene mutation sites is lowered in the LOH analysis, the K-ras gene analysis, and the like.

The present invention takes the above-mentioned problems into consideration, with an object of providing a method for detecting cancer with high accuracy through quantification of cancer cell-derived free DNA.

In order to solve the above-mentioned problems, a first aspect of the present application provides a method for detecting cancer, which includes the steps of: (1) extracting free DNA from a plasma collected from a test subject; (2) quantifying the extracted free DNA and calculating the free DNA content per unit volume of the plasma to obtain a first calculation value; (3) comparing the first calculation value with a second threshold value which is equal to or higher than a first threshold value; and (4) determining that the test subject is highly unlikely affected by cancer if the first calculation value is lower than the first threshold value, determining that the test subject is likely affected by cancer if the first calculation value is equal to or higher than the first threshold value and lower than the second threshold value, or determining that the plasma used for the quantification is contaminated by normal cell-derived DNA if the first calculation value is equal to or higher than the second threshold value.

A second aspect of the present application is a method for detecting cancer, which includes, following the step (4), the steps of: (5) if it is determined that the plasma used for the quantification is contaminated by normal cell-derived DNA, then, performing an operation for removing the normal cell-derived DNA from the plasma, extracting free DNA from the obtained plasma, quantifying the extracted DNA, and calculating the free DNA content per unit volume of the plasma after the removal operation to obtain a recalculation value, alternatively, newly collecting a plasma from the test subject, extracting free DNA therefrom, quantifying the extracted free DNA, and calculating the free DNA content per unit volume of the plasma to obtain a recalculation value, and comparing the recalculation value with the first threshold value and the second threshold value; (6) determining that the plasma used for the quantification is contaminated by normal cell-derived DNA if the recalculation value is equal to or higher than the second threshold value, and returning to the step (5); (7) determining that the test subject is highly unlikely affected by cancer if the recalculation value is lower than the first threshold value, or determining that the test subject is likely affected by cancer if the recalculation value is equal to or higher than the first threshold value and lower than the second threshold value; (8) if it is determined that the test subject is likely affected by cancer, detecting the presence/absence of mutation in the free DNA used for the determination; and (9) if a mutation is detected therein, determining that the test subject is highly likely affected by cancer of a specific organ caused by this mutation.

A third aspect of the present application may be a method for detecting cancer according to either one of the first and second aspects mentioned above, wherein the plasma is separated from a whole blood which has been mixed with a chelating agent after the collection of blood from the test subject.

A fourth aspect of the present application may be a method for detecting cancer according to the third aspect mentioned above, wherein the chelating agent is one or more types of agents selected from the group consisting of ethylenediaminetetraacetic acid, sodium ethylenediaminetetraacetate, sodium citrate, and heparin.

A fifth aspect of the present application may be a method for detecting cancer according to any one of the first through fourth aspects mentioned above, wherein the plasma is a supernatant recovered through centrifugation of a whole blood immediately after the collection of blood from the test subject, or of a whole blood which has been stored under refrigeration immediately after the collection of blood from the test subject, or a supernatant obtained by one or more times of additional centrifugation of the above-mentioned supernatant.

A sixth aspect of the present application may be a method for detecting cancer according to any one of the first through fifth aspects mentioned above, which comprises the step of concentrating the extracted free DNA, after the step (1) and before the step (2).

A seventh aspect of the invention of the present application may be a method for detecting cancer according to any one of the first through sixth aspects mentioned above, which includes the step of, if it is determined that the test subject is highly likely affected by cancer of a specific organ, further performing imaging diagnosis of the organ.

An eighth aspect of the present application may be a method for detecting cancer according to any one of the first through sixth aspects mentioned above, which includes the step of, if no mutation is detected in the free DNA, determining that the test subject is highly likely affected by cancer differing from the cancer of the specific organ.

An ninth aspect of the present application may be a method for detecting cancer according to the eighth aspect mentioned above, which includes the step of, if it is determined that the test subject is highly likely affected by cancer differing from the cancer of the specific organ, further performing imaging diagnosis of the whole body.

A tenth aspect of the present application may be a method for detecting cancer according to either one of the seventh and ninth aspects mentioned above, wherein the imaging diagnosis uses radial rays.

An eleventh aspect of the present application may be a method for detecting cancer according to any one of the second through tenth aspects mentioned above, wherein the mutation is one or more types of mutations selected from the group consisting of point mutation, microsatellite instability, and chromosomal abnormality.

A twelfth aspect of the present application may be a method for detecting cancer according to the eleventh aspect mentioned above, wherein the presence/absence of the chromosomal abnormality is detected by LOH analysis.

A thirteenth aspect of the present application may be a method for detecting cancer according to the twelfth aspect mentioned above, wherein the LOH analysis is performed with use of a microsatellite marker.

A fourteenth aspect of the present application may be a method for detecting cancer according to the twelfth aspect mentioned above, wherein the LOH analysis is performed with the use of an SNP marker.

A fifteenth aspect of the present application may be a method for detecting cancer according to the twelfth aspect mentioned above, wherein the LOH analysis is performed by comparing with DNA that is already known to be non-mutated.

A sixteenth aspect of the present application may be a method for detecting cancer according to any one of the first through fifteenth aspects mentioned above, wherein the normal cell is a lymphocyte.

A seventeenth aspect of the present application may be a method for detecting cancer according to the eleventh aspect mentioned above, wherein the target gene for detecting the presence/absence of the point mutation is K-ras gene.

An eighteenth aspect of the present application may be a method for detecting cancer according to any one of the second through seventeenth aspects mentioned above, wherein the cancer of the specific organ is lung cancer, pancreatic cancer, or breast cancer.

According to the present invention, cancer can be diagnosed with a high level of accuracy.

### BRIEF DESCRIPTION OF THE DRAWINGS

FIG. 1 is a flowchart which illustrates the steps from plasma collection to the end of the diagnosis in the present invention.

FIG. 2 is a flowchart which illustrates the step of detecting the presence/absence of mutation in DNA used for determination in the present invention.

FIG. 3 is a flowchart which illustrates a case where imaging diagnosis is further performed after the cancer diagnosis.

[A sequence listing is attached]

### BEST MODE FOR CARRYING OUT THE INVENTION

Hereunder is a detailed description of the present invention.

If the test subject is a cancer patient, their whole blood often contains cancer cell-derived free DNA leaking from disrupted cancer cells. In this case, if plasma is separated from the whole blood, the plasma contains the cancer cell-derived free DNA. Accordingly, as the cancer progresses more, that is, as the number of cancer cells increases, the number of disrupted cancer cells increases. Therefore, the cancer cell-derived free DNA content in the plasma is increased. Then, whether or not the test subject is affected by cancer can be diagnosed by quantifying the free DNA content in the plasma and checking whether or not the free DNA content in the plasma is higher than a certain predetermined threshold value. However, normal cells represented by blood cell components such as white blood cells might be disrupted depending on the preservation method of the whole blood, the separation method of the plasma, or the like.

In this case, since DNA derived from these disrupted normal cells leaks into the whole blood, the plasma contains not only the cancer cell-derived DNA but also the normal cell-derived DNA. Accordingly, highly accurate cancer diagnosis can not be done even with such a plasma.

The present invention relates to a method for detecting cancer with high accuracy by: determining whether or not apparent contamination of the plasma by normal cell-derived DNA is present; if contamination is found, removing it or newly collected plasma; quantifying the cancer cell-derived free DNA content in the plasma; and comparing the obtained quantitative value with a threshold value serving as a criterion for determining the presence/absence of the occurrence of cancer. The present invention also relates to a method for further specifying the organ where the cancer occurs, on the basis of such a highly accurate diagnosis. Hereunder is a detailed description of each step of the present invention.

(1) Firstly, in the present invention, free DNA is extracted from a plasma collected from a test subject.

The plasma for use in the present invention is separated from the whole blood, and is yielded through, for example, an operation for removing blood cell components from the whole blood. More specifically, for example, a supernatant recovered through centrifugation of the whole blood collected from the test subject, or a supernatant recovered through one or more times of additional centrifugation of the above-mentioned supernatant, may be used as the plasma. That is, the whole blood may be centrifuged and its supernatant may be recovered for use as the plasma, or an operation for further centrifuging the resultant supernatant and re-recovering its supernatant may be performed one or more times so as to use the thus recovered product as the plasma. It is normally sufficient to perform this supernatant recovery step twice, although it is more reliable for removing blood cell components to perform the step for a plurality of times more than once.

That is, blood cell components are sufficiently removed in the supernatant recovered through additional centrifugation of a supernatant which has been yielded through centrifugation of the whole blood.

Here, the supernatant component refers to a component distributed over the top of the red blood cell layer and the buffy coat after centrifugation. In addition, the condition for the centrifugation is not specifically limited as long as the blood cell components are not disrupted, and may be a condition for general use in usual centrifugation of whole blood. The blood cell components in the present invention refer to white blood cells such as lymphocytes, red blood cells, and the like.

In the present invention, it is not preferable if the blood cell components are not completely removed from the plasma because blood cell components might be disrupted during filtration of the plasma upon removal of the normal cell-derived DNA that will be described later, particularly with use of a membrane, and the plasma might be contaminated by blood cell component-derived DNA.

That is, the plasma from which the blood cell components have been more reliably removed is suitable for performing the cancer diagnosis with a higher level of accuracy.

Immediately after the blood collection, the plasma is preferably separated from the whole blood, or the whole blood is stored under refrigeration until the plasma can be separated. If the whole blood is stored without refrigeration or thawed after cryopreservation, normal cells such as the blood cell components might be disrupted. In addition, normal cells might be disrupted by vigorous agitation of the whole blood, although it can be presumed that such disruption of normal cells will not happen by shaking to an extent of usual centrifugation.

Moreover, it is suitable for the plasma separation to add a conventionally known chelating agent to the whole blood immediately after the blood collection since coagulation or the like can be prevented. This is also suitable for stable extraction of DNA since the DNase activities can be inhibited.

The chelating agent may be any blood anticoagulant for normal use in vivo or ex vivo. Specific examples thereof can include heparin, calcium ion-bindable sodium citrate, ethylenediaminetetraacetic acid (EDTA), sodium ethylenediaminetetraacetate, and an acid citrate dextrose solution. Of these, sodium citrate, EDTA, sodium ethylenediaminetetraacetate, and heparin are preferred. The chelating agent may be used either singularly or in combination of a plurality of types of agents.

The dose of these chelating agents is not specifically limited as long as the effect of the present invention is not impaired.

The method for extracting the free DNA from the plasma is not specifically limited and any conventionally known method may be used. For example, a commercially available nucleic acid extraction kit may be used.

(2) Next, in the present invention, the extracted free DNA is quantified and the free DNA content per unit volume of the plasma is calculated to obtain a first calculation value.

The method for quantifying the extracted free DNA is not specifically limited. For example, the quantification can be done by measuring the UV absorption value, or by using a fluorescent reagent which interacts with double-stranded DNA etc. and measuring its fluorescence signal. The fluorescent reagent is not specifically limited, and may be a conventionally known agent, such as fluorescein, rhodamine, acriflavine, and alexa, or a commercially available agent.

For example, unless apparent contamination of the plasma by normal cell-derived DNA is present, the first calculation value is higher in a test subject than in healthy cases if the test subject is affected by cancer, and the first calculation value is increased more as the cancer progresses more.

In the present invention, the free DNA extracted from the plasma may be concentrated. That is, after the step of extracting free DNA from a plasma collected from a test subject, a step of concentrating the extracted free DNA may be performed, before performing the step of quantifying the extracted free DNA and calculating the free DNA content per unit volume of the plasma to obtain a first calculation value. By doing so, the first calculation value can be accurately obtained.

Since the free DNA extracted from the plasma is usually in a dissolved state in an aqueous or other solution, the method for concentrating the free DNA can be specifically exemplified by a method for removing a part of the solvent, and a method for taking out the free DNA from the solution and then re-dissolving it at an appropriate concentration with water or other solvent.

The method for removing a part of the solvent can be exemplified by a filtration method with use of a reverse osmotic membrane (RO membrane) etc. In addition, it may also be exemplified by a reduced-pressure drying method with use of a centrifugal concentrator such as a SpeedVac. The method for extracting the free DNA can be exemplified by a method for adding an organic solvent such as ethanol to the free DNA solution to precipitate the free DNA, and then extracting it by filtration or the like. The extracted free DNA may be re-dissolved in a solvent suitable for the quantification.

(3) Next, in the present invention, the first calculation value is compared with a second threshold value which is equal to or higher than a first threshold value.

Here, the first threshold value refers to a value to determine whether or not the test subject is affected by cancer.

The second threshold value refers to a value to determine whether or not apparent contamination of the plasma by normal cell-derived DNA is present. The first threshold value and the second threshold value normally vary depending on the type of cancer, and thus need to be appropriately set. For example, the cancer cell-derived DNA contents per unit volume of plasma are previously examined on preferably as many healthy subjects and cancer patients as possible, and a DNA content capable of the most clear discrimination between the healthy subjects and cancer patients can be set as the first threshold value. Then, the upper limit of the first threshold value is estimated from the cancer cell-derived DNA content per unit volume of the plasma of the cancer patients, and a value slightly over the upper limit can be set as the second threshold value. For example, in cases of breast cancer, lung cancer, pancreatic cancer, and the like, it is preferable to set the first threshold value at about 10 ng per 1 ml of plasma, and the second threshold value at about 100 ng per 1 ml of plasma.

If the first calculation value is equal to or higher than the first threshold value and lower than the second threshold value, it shows that the plasma used for the quantification contains more cancer cell-derived free DNA than that of healthy subjects and that no apparent contamination of the plasma by normal cell-derived DNA is found. (4) In this case, in the present invention, it is determined that the test subject is likely affected by cancer (hereunder, may be abbreviated as Determination A).

(4) On the other hand, if the first calculation value is equal to or higher than the second threshold value, in the present invention, it is determined that the plasma used for the quantification is contaminated by normal cell-derived DNA (hereunder, may be abbreviated as Determination B).

As described above, the reason why this first calculation value is higher than the second threshold value is that, for example, normal cells are disrupted and the resultant normal cell-derived DNA leaks into the whole blood to thereby contaminate the plasma separated from the whole blood. Otherwise, depending on how the operation has been done, the reason might be such that the normal cells in the plasma are disrupted and the resultant normal cell-derived DNA contaminates the plasma during the time until the extracted free DNA is quantified. Here, the normal cells easily serving as the causative factor of such DNA contamination can be exemplified by blood cell components such as white blood cells, and in particular, lymphocytes.

In addition, if the first calculation value is lower than the first threshold value, it shows that the plasma used for the quantification contains equivalent or lower cancer cell-derived free DNA than that of healthy subjects and that no apparent contamination of the plasma by normal cell-derived DNA is found. In this case, in the present invention, it is determined that the test subject is highly unlikely to be affected by cancer (hereunder, may be abbreviated as Determination C).

As described before, in the case of the Determination B, accurate cancer diagnosis can not be performed. Therefore, in the present invention, (5) in the case of the Determination B, subsequently, it is preferable to newly collect a plasma from the test subject, extract free DNA, quantify the extracted free DNA, and calculate the free DNA content per unit volume of the plasma so as to obtain a recalculation value (hereunder, abbreviated as the retrial operation). If the causative factor of the Determination B has happened due to the operation before the quantification of the extracted free DNA, the recalculation value might be lower than the second threshold value.

(5) Alternatively, in the case of the Determination B, instead of the retrial operation, an operation for removing the contaminant normal white blood cell-derived DNA from the plasma may be performed before the extraction of free DNA from the obtained plasma, quantification of the extracted DNA, and calculation of the free DNA content per unit volume of the plasma after the removal operation in the same manner as described above so as to obtain a recalculation value. Usually, normal white blood cell-derived DNA has a molecular size of at least 10000 by (that is, unfragmented), whereas cancer cell-derived DNA is fragmented into smaller sizes of base pairs. Accordingly, the normal cell-derived DNA can be removed from the plasma by utilizing this molecular size difference. The removal method may be any method as long as this molecular size difference can be utilized. Specific examples thereof can include plasma filtration methods with use of membranes such as an ultrafiltration membrane (UF membrane), a microfiltration membrane (MF membrane), or other membranes, and plasma filtration methods with use of agarose or other gel slices. Moreover, a publicly known suitable method may be applied according to the membrane etc. to be used. In addition, such a DNA separation can use various commercially available membranes and gel slices. If a gel slice is used, an Ultrafree-DA Filter Device (Product Name, manufactured by Millipore) for example, which can remove nucleic acids of 10000 by or longer is suitable. Moreover, even without a gel slice, fractioning can be performed on a molecular size basis by a similar filtration method.

Subsequently, the obtained recalculation value is compared with the first threshold value and the second threshold value (hereunder, abbreviated as the re-comparison).

Normally, if the normal cell disruption in the plasma is avoided through the retrial operation, or if the normal cell-derived DNA is removed from the plasma through the removal operation, the recalculation value will be lower than the first calculation value. However, normal cells might be disrupted even in the retrial operation for example, or the normal cell-derived DNA removal rate might be lowered depending on, for example, the filtration method in the removal operation. Accordingly, the recalculation value might be higher than the second threshold value even with a one-off retrial or removal operation. (6) In this case, it is determined that the plasma used for the quantification is contaminated by normal cell-derived DNA, and the flow goes back to the step (5). That is, the retrial/removal operation and the re-comparison are performed again. In this case, the retrial/removal operation and the re-comparison may be performed as many times as required until the recalculation value becomes lower than the second threshold value. However, if the recalculation value will not be lower than the second threshold value even through a plurality of repetitions, another diagnosis method may be jointly used.

If the recalculation value is lower than the second threshold value, the determination of the recalculation value may be performed in the same manner as that of the determination of the first calculation value mentioned above.

That is, if the recalculation value is equal to or higher than the first threshold value and lower than the second threshold value, it shows that the plasma used for the quantification contains more cancer cell-derived free DNA than that of healthy subjects and that no apparent contamination of the plasma by normal cell-derived DNA is found. (7) In this case, in the present invention, it is determined that the test subject is likely affected by cancer (hereunder, may be abbreviated as Determination BA).

On the other hand, if the recalculation value is lower than the first threshold value, in the present invention, it is determined that the test subject is highly unlikely to be affected by cancer (hereunder, may be abbreviated as Determination BC).

(8) In the present invention, if it is determined that the test subject is likely affected by cancer, subsequently, the presence/absence of mutation in the free DNA used for the determination is detected. Here, the mutation is not specifically limited as long as it can serve as a causative factor of cancer onset. Specific examples thereof can include point mutation, microsatellite instability, and chromosomal abnormality. In addition, a plurality of types of mutations may be targeted in the detection of the presence/absence of mutation.

The detection of the presence/absence of mutation in DNA may be performed by a conventionally known method, and is not specifically limited. For example, when it comes to the detection of the presence/absence of chromosomal abnormality, LOH analysis, and so on is suitable. In addition, the LOH analysis can be performed with use of various types of markers such as a microsatellite marker and a SNP marker. The presence/absence of mutation can be efficiently detected by using such a marker. Here, SNP stands for Single Nucleotide Polymorphism. Moreover, the LOH analysis may also be performed by using DNA that is already known to be non-mutated to compare their nucleotide sequences.

The method for detecting the presence/absence of mutation may be used either singularly or in combination of a plurality of methods.

Since the DNA mutation varies depending on the type of cancer, the detection of the presence/absence of mutation is performed after selecting the target DNA according to the type of suspected cancer or the type of cancer to be checked.

For example, since it is known that point mutations of the K-ras gene occur in about 70% of pancreatic cancers, it is preferable to detect the presence/absence of point mutation in the K-ras gene in order to highly accurately diagnose whether or not the test subject is affected by pancreatic cancer. In addition, deletions and amplifications in specific chromosome arms to various types of cancers have been reported in LOH analyses. For example, frequent deletions in the short arm of chromosome 3 in lung cancer are known, while multiplication of the long arm of chromosome 8 and RB2 amplification in breast cancer are known. Accordingly, these LOH analyses may be performed in accordance with the type of cancer.

The free DNA extracted from the plasma may be directly provided to the detection of the presence/absence of mutation, although it is preferable to perform a highly accurate detection to amplify the extracted free DNA before providing it to the detection of the presence/absence of mutation. The DNA amplification may be performed by a conventionally known method such as the polymerase chain reaction (hereunder, abbreviated as PCR) method.

For example, in the PCR method, primers can be selected so as to amplify the target DNA in which the presence/absence of mutation is to be detected, so that the presence/absence of mutation in this amplified product is detected.

(9) Next, in the present invention, if a mutation is detected in the extracted free DNA, it is determined that the test subject is highly likely affected by cancer of a specific organ caused by this mutation. The reason is that, since it has been determined that the test subject is likely affected by cancer, and mutation has been detected in the DNA, this determination can be attributed to this mutation.

On the other hand, if no mutation is detected, it is preferable to determine that the test subject is highly likely affected by cancer differing from the specific organ. The reason is that, if no mutation has been detected in the DNA regardless of the determination that the test subject is likely affected by cancer, it can be attributed to the fact that inappropriate DNA was used for the detection of the presence/absence of mutation and other DNA is mutated.

In the present invention, if it is determined that the test subject is likely affected by cancer (determinations A and BA), it is preferable to further perform an imaging diagnosis. Here, the imaging diagnosis may be a conventionally known one, and can be exemplified by the X-ray examination, the magnetic resonance imaging (MRI) examination, the computed tomography (CT) examination, the radioisotope (RI) examination, and the endoscopic ultrasonography examination. The imaging diagnosis may be performed either singularly or in combination of a plurality of types. It is preferable to combine a plurality of types since the diagnosis can be performed with even higher accuracy. In addition, it is preferable to appropriately select the type of the method for the imaging diagnosis according to the purpose, as described below.

If it is determined that the test subject is highly likely affected by cancer differing from the specific organ, the site of cancer occurrence can be specified by jointly performing the imaging diagnosis. In this case, the imaging diagnosis is preferably performed over a wide range, and more preferably over the whole body. By so doing, it is sufficient not only for specifying the site of cancer occurrence but also for checking the presence/absence of cancer metastasis. In addition, if the site of diagnosis covers a wide range in this manner, the imaging diagnosis is preferably performed by MRI examination, CT examination, or the like, which can precisely analyze wide regions.

On the other hand, even if it is determined that the test subject is highly likely affected by cancer of a specific organ, it is preferable to jointly perform the imaging diagnosis. The reason is that, even if the organ having an occurrence of cancer is specified, it is not possible to specify the site of the organ in which there is an occurrence of cancer. In this case, the imaging diagnosis is preferably performed by intensively targeting the organ suspected of having an occurrence of cancer.

The presence/absence of cancer metastasis can also be checked by targeting nearby organs. If the site of diagnosis is limited within such small areas, any imaging diagnosis method is suitable, and may be appropriately selected according to the type of the organ being the diagnosis target, although it is more preferable to employ helical CT examination, X-ray examination, endoscopic ultrasonography examination, and the like. In addition, since the diagnosis site is small, for example, the exposure dose of radial rays can be reduced in examinations such as the X-ray examination, the CT examination, and the RI examination which involve irradiation of radial rays, and burdens on the test subject can be further alleviated.

If it is determined that the test subject is highly unlikely affected by cancer (determinations C and BC), the step of detecting the presence/absence of mutation in the free DNA extracted from the plasma of the test subject need not be performed.

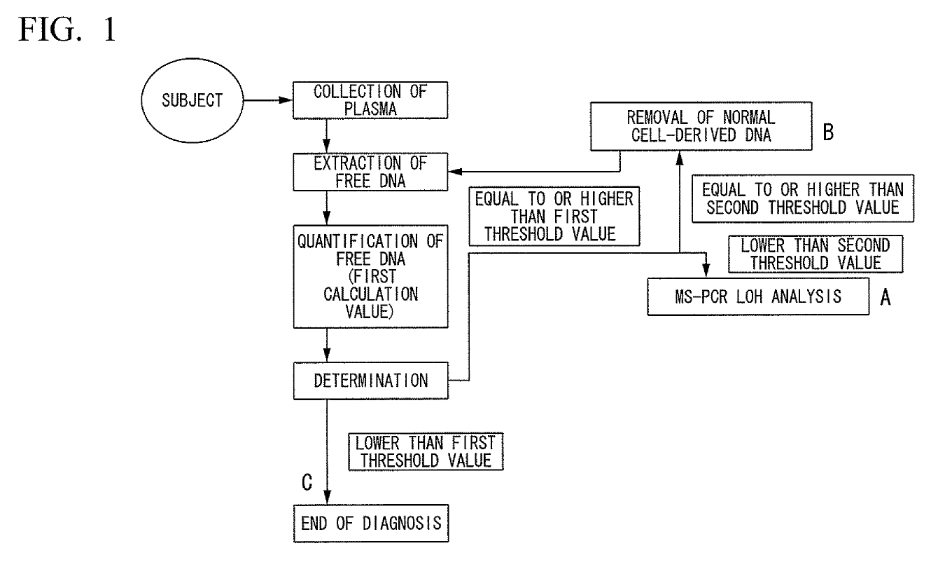

Hereunder is a description of the method for detecting cancer of the present invention with specific examples referring to FIGS. 1 to 3.

FIG. 1 is a flowchart which illustrates the steps from the plasma collection to the end of the diagnosis. Free DNA is extracted from a plasma collected from a test subject, the extracted free DNA is quantified, and the free DNA content per unit volume of the plasma is quantified to obtain the first calculation value. If the first calculation value is lower than the first threshold value, it is determined that the test subject is highly unlikely to be affected by cancer and the diagnosis may be ended (step of Determination C, hereunder, abbreviated as step C). On the other hand, if the first calculation value is equal to or higher than the first threshold value and lower than the second threshold value, it is determined that the test subject is likely affected by cancer, and the flow goes to the step of detecting the presence/absence of mutation in the DNA used for the determination (step of Determination A, hereunder, abbreviated as step A). If the first calculation value is equal to or higher than the second threshold value, it is determined that the plasma used for the quantification is contaminated by normal cell-derived DNA, the above-mentioned retrial or removal operation is performed to remove the normal cell-derived DNA, and then the free DNA content per unit volume of the plasma is again quantified to obtain the recalculation value (step of Determination B, hereunder, abbreviated as step B). If the recalculation value is less than the second threshold value, the flow goes to the step A or C. If the recalculation value is equal or higher than the second threshold value, the step B is repeated before going to the step A or C.

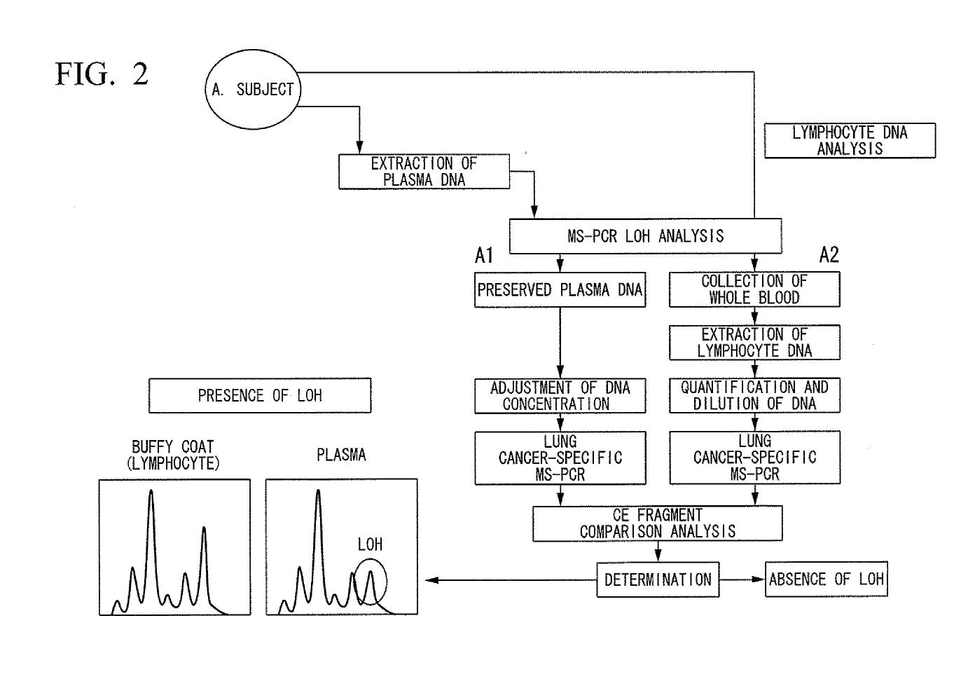

FIG. 2 is a flowchart which illustrates the step of detecting the presence/absence of mutation in the DNA used for the determination according to the step A. Specifically, it illustrates the step of detecting the presence/absence of chromosomal abnormality caused by lung cancer through LOH analysis of the short arm of chromosome 3 with use of a microsatellite marker.

For use in the determination, preserved free DNA that has been collected and extracted from the test subject is prepared into a solution by adjusting the concentration of preserved free DNA. This DNA is used as the template and primers are selected for amplifying the target nucleotide sequence in which the presence/absence of mutation is to be detected, followed by an amplification reaction through PCR (microsatellite PCR) (step A1). Meanwhile, the whole blood is collected from the test subject, lymphocytes serving as the normal cells are separated therefrom, and DNA is extracted from these lymphocytes. The lymphocyte-derived DNA in the solution is quantified and diluted, and the DNA is used as the template, followed by an amplification reaction through microsatellite PCR in the same manner as mentioned above (step A2). Next, comparison is made between the plasma-derived free DNA and the lymphocyte-derived DNA, regarding the fragment patterns of the obtained amplification products by capillary electrophoresis or the like to detect the presence/absence of LOH in the plasma-derived free DNA. If apparent change in the signal pattern (lowering in the signal intensity) is found in the plasma-derived free DNA, LOH occurs and thus it is determined that the test subject is highly likely affected by cancer of a specific organ. On the other hand, if no apparent difference in the signal pattern is found between the plasma-derived free DNA and the lymphocyte-derived DNA, it is preferable to determine that the test subject is highly likely affected by cancer differing from the specific organ caused by a chromosomal abnormality of the detection target.

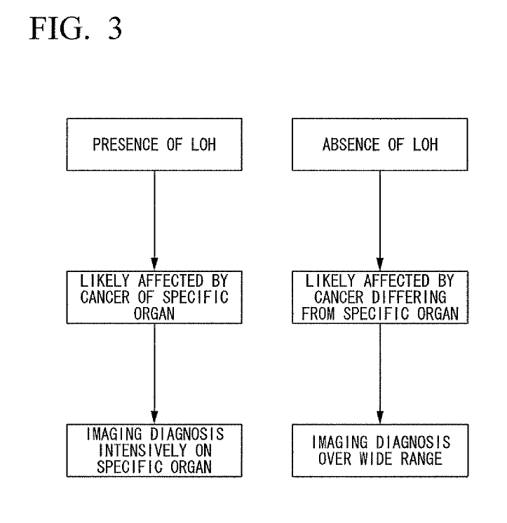

FIG. 3 is a flowchart which illustrates a case where the imaging diagnosis is further performed after the cancer diagnosis according to the step of FIG. 2. If LOH is detected, the occurrence of cancer in a specific organ is suspected, and thus it is preferable to perform the imaging diagnosis intensively on the target organ. For example, if lung cancer is suspected, chest imaging diagnosis by helical CT, chest X-ray examination, or the like is suitably performed. On the other hand, if LOH is not detected, the organ of cancer occurrence can not be specified, and thus the imaging diagnosis is preferably performed over a variety of places of a patient's body.

The present invention is suitable for types (organs) of cancers in which the DNA derived from cancer cell of a specific organ is frequently observed in the whole blood of the cancer patient. Specific examples thereof can include lung cancer, pancreatic cancer, and breast cancer. On the other hand, the present invention is not suitable for colon cancer, gastric cancer, and the like.

As described above, according to the method for detecting cancer of the present invention, the effect of apparent contamination of plasma by normal cell-derived DNA can be eliminated so that a highly accurate quantitative value of the cancer cell-derived free DNA content can be compared with a specific threshold value, by which highly accurate cancer diagnosis can be performed.

### Example

Hereunder is a more detailed description of the present invention with reference to a specific Example. However, the present invention is not to be considered as being limited by the following Example. In addition, in the following Example, the term “/ml plasma” refers to “per 1 ml of plasma”.

### (DNA Quantification)

Plasma from thirteen test subjects was collected at 1 ml each (sample Nos. 1 to 13), then frozen and thawed. Nucleic acid was extracted from the plasma according to a publicly known technique using the QIAamp DNA Blood Midi Kit (Product Name, manufactured by Qiagen). The extraction was performed using 100 μL of Buffer AE (Product Name, manufactured by Qiagen) provided in this kit. After the extraction, the extracted nucleic acid was quantified according to a publicly known technique using the PicoGreen dsDNA Quantitation Kit (Product Name, manufactured by Invitrogen). Based on this quantitative value, the free DNA content per 1 ml of plasma (first calculation value) was calculated. The first threshold value was set at 10 ng/ml plasma and the second threshold value was set at 100 ng/ml plasma. That is, the setting was such that: if the first calculation value was less than 10 ng/ml plasma, it was determined that the test subject was highly unlikely affected by cancer (Determination C); if the first calculation value was equal to or higher than 10 ng/ml plasma and equal to or lower than 100 ng/ml plasma, it was determined that the test subject was likely affected by cancer (Determination A); and if the first calculation value was higher than 100 ng/ml plasma, it was determined that the plasma was contaminated by normal cell-derived DNA (Determination B).

The calculation results of their free DNA contents are shown in Table 1. Since sample Nos. 7 and 13 showed higher first calculation values than the second threshold values, it was determined that their plasmas were contaminated by normal cell-derived DNA. Then, DNA fragments of 100 to 10000 by were removed from sample Nos. 7 and 13 according to a publicly known procedure using the Ultrafree-DA Filter Device (Product Name, manufactured by Millipore). After the removal, the samples were again subjected to the quantification of nucleic acid using the PicoGreen dsDNA Quantitation Kit. Based on each quantitative value, the free DNA content per 1 ml of plasma (recalculation value) was calculated. The results are shown in Table 1. As shown in Table 1, the recalculation value of sample No. 7 was equal to or higher than the first threshold value and lower than the second threshold value (Determination A), while the recalculation value of sample No. 13 was less than the first threshold value (Determination C).

The determination results of sample Nos. 1 to 13 are as follows.

Since sample Nos. 1 to 3 and sample Nos. 9 to 12 showed lower first calculation values than the first threshold values (Determination C), and sample No. 13 showed a lower recalculation value than the first threshold value (Determination BC), it was determined that the test subjects who donated these samples were highly unlikely affected by cancer. In fact, the test subjects who donated sample Nos. 1 to 3 were all healthy subjects and unaffected by cancer, and the test subjects who donated sample Nos. 10 to 13 were all patients with benign tumors and unaffected by cancer. All of these test subjects were not cancer patients. On the other hand, the test subject who donated sample No. 9 was in fact a cancer patient.

In addition, since the sample Nos. 4 to 6, and 8 showed the first calculation values equal to or higher than the first threshold values and lower than the second threshold values (Determination A), and the sample No. 7 showed the recalculation value equal to or higher than the first threshold value and lower than the second threshold value (Determination BA), then it was determined that the test subjects who donated these samples were likely affected by cancer. In fact, the test subjects who donated the sample Nos. 4 to 6, and 8 were cancer patients.

From the above results, the accuracy of the cancer diagnosis according to the diagnosis method of the present invention (number of true diagnoses/number of test subjects×100) was 92% and the precision of cancer patients (number of true cancer diagnoses/number of cancer patients×100) was 83%.

TABLE 1

| | Free DNA content in plasma | | |
|---|---|---|---|
| | (ng/ml plasma) | | |
| | First calculation | | |
| Sample No. | value | Recalculation value | Clinical finding |
| 1 | 1.50 | — | Normal |
| 2 | 1.10 | — | Normal |
| 3 | 2.50 | — | Normal |
| 4 | 15.20 | — | Cancer |
| 5 | 12.30 | — | Cancer |
| 6 | 22.70 | — | Cancer |
| 7 | 109.60 | 2.40 | Cancer |
| 8 | 26.50 | — | Cancer |
| 9 | 7.90 | — | Cancer |
| 10 | 1.50 | — | Benign tumor |
| 11 | 2.30 | — | Benign tumor |
| 12 | 3.40 | — | Benign tumor |
| 13 | 144.7 | 3.2 | Benign tumor |

### (Point Mutation Analysis)

Next, sample Nos. 4 to 8, for which it was determined that the donor test subjects were likely affected by cancer, were subjected to point mutation analysis of the K-ras codons 12 and 13.

First, PCR-amplified fragments were prepared using DNA extracted from sample Nos. 4 to 8 as the template, and using a set of a forward primer comprising the nucleotide sequence of SEQ ID NO: 1 and a reverse primer comprising the nucleotide sequence of SEQ ID NO: 2 which were each labeled with rhodamine as the K-ras cancer gene primer. That is, 0.5 μl of TaKaRa Taq (5 U/μl) (manufactured by Takara Bio), 8 μl of dNTP mixture (2.5 mM each), 5 ng of the template DNA, and 2 μl of the forward and reverse primers (20 pmol/μl each) were mixed, and sterile distilled water was added thereto until the liquid volume reached 100 μl. Then, the mixture was set in a thermal cycler, and subjected to PCR amplification through 25 cycles of the heating condition of “94° C. for 30 seconds, 55° C. for 2 minutes, and 72° C. for one minute”.

Next, 1 μl of the PCR reaction solution was 30-fold diluted with a solution of “95% formamide, 20 mM EDTA”. 1 μl of this dilution was electrophoresed under the following condition, and was subjected to the detection of the fluorescence signal using the Fluorescence Bio Imaging Analyzer FMBIO System (Product Name, manufactured by Takara Bio). The presence/absence of mutation in the DNA extracted from each sample was checked by concurrently flowing the normal type control PCR amplified fragments as the marker. The analysis results are shown in Table 2.

### <Electrophoresis Condition>

Buffer solution: 0.5×TBE, Gel: 5% polyacrylamide gel (1% cross-linked in 0.5×TBE) 160×450×0.35 (mm), Temperature: 4° C., Dye marker: 30 W constant loading buffer (xylene cyanol and bromophenol blue)

As a result of the analysis, mutations of the K-ras gene were found in the DNA extracted from samples Nos. 5, 7, and 8. Therefore it was determined that the test subjects who donated these samples were highly likely affected by pancreatic cancer. In fact, these test subjects were patients with pancreatic cancer.

On the other hand, mutations of the K-ras gene were not found in the DNA extracted from the samples Nos. 4 and 6. Therefore it was determined that the test subjects who donated these samples were highly likely affected by cancer differing from pancreatic cancer. In fact, the test subject who donated sample No. 4 was a patient with breast cancer, and the test subject who donated sample No. 6 was a patient with lung cancer.

TABLE 2

| | Free DNA content in | | Result of K-ras | |
|---|---|---|---|---|
| | plasma (ng/ml plasma) | | analysis, | |
| | First | | Presence/ | |
| Sample | calculation | Recalculation | Absence of | |
| No. | value | value | mutation | Clinical finding |
| 4 | 15.20 | — | Absence | Breast cancer |
| 5 | 12.30 | — | Presence | Pancreatic cancer |
| 6 | 22.70 | — | Absence | Lung cancer |
| 7 | 109.60 | 20.40 | Presence | Pancreatic cancer |
| 8 | 26.50 | — | Presence | Pancreatic cancer |
| 9 | 7.90 | — | — | Pancreatic cancer |

(LOH Analysis with Microsatellite Marker)

For sample Nos. 4 to 9, an attempt was made to detect lung cancer through the LOH analysis with use of a microsatellite marker by the following procedure.

First, PCR-amplified fragments were prepared using 2 μg of DNA extracted from sample Nos. 4 to 9 as the template, and using a forward primer comprising the nucleotide sequence of SEQ ID NO: 3 and a reverse primer comprising the nucleotide sequence of SEQ ID NO: 4 as the primer for detecting D3S1234. These primers were a pair of GeneScan fluorescent primers, being the GeneScan fluorescent tailed primer pair (manufactured by Applied Biosystems JAPAN, Nos. 450056 and 4304979).

That is, 2 μl of 10× Ex Taq buffer, 0.1 μl of Ex Taq (manufactured by Takara Bio, #RR001A), 1.6 μl of dNTP mixture (2.5 mM each), 1 μl of the template DNA (5 ng/μl), 1 μl of the forward and reverse primers (5 μl/l each), and 13.3 μl of sterile distilled water were mixed for use as the PCR reaction solution. The solution was set in a thermal cycler, and subjected to PCR amplification through heat denaturation at 94° C. for 5 minutes, 30 cycles of the heating condition of “94° C. for 30 second, 55° C. for 30 seconds, and 72° C. for 30 seconds”, and final extension at 72° C. for 5 minutes.

Next, in a sole-use 96-well plate were previously dispensed Hi-Di Formanide (Product Name, #4311320, manufactured by Applied Biosystems JAPAN) and GeneScan-500 ROX STANDARD mix (Product Name, #401734 manufactured by Applied Biosystems JAPAN) at 10 μl each, and the PCR reaction solution was dispensed therein at 1 μl each. Then, the mixture was subjected to heat denaturation treatment under the heating condition of 95° C. for 5 minutes in a thermal cycler, and immediately thereafter the plate was left still in ice for 5 minutes or longer. Next, according to the procedure manual of ABI3100, the heat-denaturated samples were subjected to capillary electrophoresis. The electrophoresed data analysis was performed by GeneScan 3.7 (Product Name, manufactured by Applied Biosystems JAPAN).

As a result of the analysis, a deletion of the microsatellite marker located at 3p21.1-3p14.2 on the short arm of chromosome 3 was found in the DNA extracted from sample No. 6. This deletion is often found in lung cancer. In fact, the clinical finding had presumed sample No. 6 as a lung cancer sample. Therefore, the LOH analysis result was effective for specifying lung cancer.

### INDUSTRIAL APPLICABILITY

The present invention can be used for cancer diagnosis in the medical field.

1. A method for detecting cancer, which comprises the steps of:
(1) extracting free DNA from a plasma collected from a test subject;
(2) quantifying the extracted free DNA and calculating the free DNA content per unit volume of the plasma to obtain a first calculation value;
(3) comparing the first calculation value with a second threshold value which is equal to or higher than a first threshold value; and
(4) determining that the test subject is highly unlikely affected by cancer if the first calculation value is lower than the first threshold value, determining that the test subject is likely affected by cancer if the first calculation value is equal to or higher than the first threshold value and lower than the second threshold value, or determining that the plasma used for the quantification is contaminated by normal cell-derived DNA if the first calculation value is equal to or higher than the second threshold value.

2. A method for detecting cancer, according to claim 1 further comprises, following said step (4), the steps of:
(5) if it is determined that the plasma used for the quantification is contaminated by normal cell-derived DNA, then, performing an operation for removing the normal cell-derived DNA from the plasma, extracting free DNA from the obtained plasma, quantifying the extracted DNA, and calculating the free DNA content per unit volume of the plasma after said removal operation to obtain a recalculation value, alternatively, newly collecting a plasma from the test subject, extracting free DNA therefrom, quantifying the extracted free DNA, and calculating the free DNA content per unit volume of the plasma to obtain a recalculation value, and comparing the recalculation value with said first threshold value and said second threshold value;
(6) determining that the plasma used for the quantification is contaminated by normal cell-derived DNA if said recalculation value is equal to or higher than the second threshold value, and returning to said step (5);
(7) determining that the test subject is highly unlikely affected by cancer if said recalculation value is lower than the first threshold value, or determining that the test subject is likely affected by cancer if said recalculation value is equal to or higher than the first threshold value and lower than the second threshold value;
(8) if it is determined that the test subject is likely affected by cancer, the presence/absence of mutation in the free DNA used for the determination; and
(9) if a mutation is detected therein, determining that the test subject is highly likely affected by cancer of a specific organ caused by this mutation.

3. The method for detecting cancer according to claim 1 or claim 2, wherein said plasma is separated from a whole blood which has been mixed with a chelating agent after the blood collection from the test subject.

4. The method for detecting cancer according to claim 3, wherein said chelating agent is one or more types of agents selected from the group consisting of ethylenediaminetetraacetic acid, sodium ethylenediaminetetraacetate, sodium citrate, and heparin.

5. The method for detecting cancer according to claim 1 or claim 2, wherein said plasma is a supernatant recovered through centrifugation of a whole blood immediately after the blood collection from the test subject, or of a whole blood which has been stored under refrigeration immediately after the blood collection from the test subject, or a supernatant obtained by one or more times of additional centrifugation of the abovementioned supernatant.

6. The method for detecting cancer according to claim 1 or claim 2, which comprises the step of concentrating the extracted free DNA, after said step (1) and before said step (2).

7. The method for detecting cancer according to claim 1, which comprises the step of, if it is determined that the test subject is highly likely affected by cancer of a specific organ, further performing imaging diagnosis of the organ.

8. The method for detecting cancer according to claim 1 or claim 2, which comprises the further step of, if no mutation is detected in the free DNA, determining that the test subject is highly likely affected by cancer differing from said cancer of the specific organ.

9. The method for detecting cancer according to claim 8, which comprises the further step of, if it is determined that the test subject is highly likely affected by cancer differing from said cancer of the specific organ, further performing imaging diagnosis of the whole body.

10. The method for detecting cancer according to claim 7, wherein said imaging diagnosis uses radial rays.

11. The method for detecting cancer according to claim 2, wherein said mutation is one or more types of mutations selected from the group consisting of point mutation, microsatellite instability, and chromosomal abnormality.

12. The method for detecting cancer according to claim 11, wherein the presence or absence of said chromosomal abnormality is detected by LOH analysis.

13. The method for detecting cancer according to claim 12, wherein said LOH analysis is performed with use of a microsatellite marker.

14. The method for detecting cancer according to claim 12, wherein said LOH analysis is performed with use of a SNP marker.

15. The method for detecting cancer according to claim 12, wherein said LOH analysis is performed by comparing with DNA that is already known to be non-mutated.

16. The method for detecting cancer according to claim 1 or claim 2, wherein said normal cell is a lymphocyte.

17. The method for detecting cancer according to claim 11, wherein the target gene for detecting the presence or absence of said point mutation is K-ras gene.

18. The method for detecting cancer according to claim 2, wherein said cancer of the specific organ is lung cancer, pancreatic cancer, or breast cancer.

19. The method for detecting cancer according to claim 2, which comprises the step of, if it is determined that the test subject is highly likely affected by cancer of a specific organ, further performing imaging diagnosis of the organ.

20. The method for detecting cancer according to claim 19, wherein said imaging diagnosis uses radial rays.

21. The method for detecting cancer according to claim 9, wherein said imaging diagnosis uses radial rays.


SEQUENCE LISTING (4 sequences)

SEQ ID NO: 1 - DNA, 20 bp, Artificial Sequence
  Designed DNA fragment to act as a primer in PCR method for amplification of K-ras gene.
gactgaatataaacttgtgg

SEQ ID NO: 2 - DNA, 20 bp, Artificial Sequence
  Designed DNA fragment to act as a primer in PCR method for amplification of K-ras gene.
ctattgttggatcatattcg

SEQ ID NO: 3 - DNA, 20 bp, Artificial Sequence
  Designed DNA fragment to act as a primer in PCR method for amplification of DNA fragment _gD3S1234_h
cctgtgagacaaagcaagac

SEQ ID NO: 4 - DNA, 20 bp, Artificial Sequence
  Designed DNA fragment to act as a primer in PCR method for amplification of DNA fragment _gD3S1234_h
gacattaggcacagggctaa